(19) United States
(12) Patent Application Publication
(10) Pub. No.: US 2015/0297125 A1
(43) Pub. Date: Oct. 22, 2015

(54) OXIMETRY SENSOR ASSEMBLY AND METHODOLOGY FOR SENSING BLOOD OXYGEN CONCENTRATION

(71) Applicant: FAURECIA AUTOMOTIVE SEATING, LLC, Troy, MI (US)

(72) Inventors: Sean M. MONTGOMERY, Astoria, NY (US); Matthew K. BENSON, Holland, MI (US); David L. CUMMINGS, Jackson Heights, NY (US); Pioter DRUBETSKOY, Bronx, NY (US); Jeffery T. BONK, Chesterfield, MI (US)

(21) Appl. No.: 14/443,458

(22) PCT Filed: Nov. 26, 2013
(86) PCT No.: PCT/US13/71884

§ 371 Date: May 18, 2015

Related U.S. Application Data
(60) Provisional application No. 61/730,374, filed on Nov. 27, 2012.

(51) Int. Cl. A61B 5/1455; A61B 5/1495; A61B 5/18; A61B 5/145; A61B 5/00

(52) U.S. Cl. CPC A61B 5/14551; A61B 5/14546; A61B 5/7275; A61B 5/18; A61B 5/1495; A61B 5/6893

(57) ABSTRACT

A sensor in accordance with the present disclosure is configured to measure a subject's blood oxygen concentration. In illustrative embodiments, the sensor may be coupled to a seat for use in a seating environment or any other suitable environment. In illustrative embodiments, the sensor is an oximetry sensor assembly being provided in a vehicle seat or other support in proximity and contact with an occupant's body.

### PRIORITY CLAIM

This application incorporates by reference and claims the benefit of U.S. Provisional Patent Application Ser. No. 61/730,374 filed Nov. 27, 2012, which is expressly incorporated by reference herein.

### BACKGROUND

The present disclosure relates to a sensor configured to sense a physiological attribute of a human, in particular a human seated on a seat such as, for example, a vehicle seat in a vehicle. More particularly, the present disclosure relates to an oximetry sensor assembly and a methodology for using the sensor assembly to measure a subject's blood oxygen content.

### SUMMARY

A sensor in accordance with the present disclosure is configured to measure a subject's blood oxygen concentration. In illustrative embodiments, the sensor may be coupled to a seat for use in a seating environment or any other suitable environment. In illustrative embodiments, the sensor is an oximetry sensor assembly being provided in a vehicle seat, a hospital bed, a wheel chair, or other support in proximity and contact with an occupant's body.

In illustrative embodiments, the sensor includes oxygen-measurement means for measuring a subject's blood oxygen content through one ore more layers of clothing without knowing the number of clothing layers or what material each layer is made from. The oxygen-measurement means includes a variable-output light source and process means for incrementally changing an amount of light emitted from the variable-output light source until a measurement can be taken from the subject.

In illustrative embodiments, the sensor is provided in one or more locations within an occupant-support base located in a vehicle and formed to include a seat bottom and a seat back extending upwardly from the seat bottom. For example, the sensor is provided in a seat bottom that includes trim and a cushion in a cushion-receiving space defined by that cushion cover.

In illustrative embodiments, a sensor assembly for measuring a subject's blood oxygen concentration comprises light-emission means for emitting light at least one wavelength, the light reflecting from the subject's body through at least one layer of clothing, light-receiver means for receiving the reflected light from the subject's body, means for analyzing the amount of reflected light to determine the blood oxygen concentration of the subject, and means for calibrating the light-emission means to take into consideration the presence of the at least one layer of clothing.

According to a further embodiment of the present disclosure, the means for analyzing the amount of reflected light determines an amount of light absorbed by the subject's body to determine the blood oxygen concentration of the subject.

According to a further embodiment of the present disclosure, the means for analysing the amount of reflected light detects the blood oxygen concentration to predict Fat Embolism Syndrome (FES), Hypoxemia, or a fracture complication.

According to a further embodiment of the present disclosure, the means for analyzing the amount of reflected light from the subject's body does so using reflectance pulse oximetry.

According to a further embodiment of the present disclosure, the means for calibrating the light-emission means identifies at least one optimum level and/or at least one type of emitted light for emission by the light-emission means.

According to a further embodiment of the present disclosure, the means for calibrating the light-emission means performs calibration every time that a subject comes in contact with a sensor located in the seating environment in the motor vehicle.

According to a further embodiment of the present disclosure, the light-receiver means is a photodetector that measures an amount of light reflected from the subject's body.

According to a further embodiment of the present disclosure, the sensor assembly further comprises circuitry coupled to the photodetector for buffering and filtering of a photodetector output signal and at least one operational amplifier that establishes a virtual ground and buffering and filtering of the photodetector output signal.

According to a further embodiment of the present disclosure, the sensor assembly further comprises at least one operational amplifier that establishes a virtual ground for buffering and filtering of the photodetector output signal.

According to a further embodiment of the present disclosure, the output signal of the photodetector is coupled to the means for calibrating the light-emission means and performs analysis of the output signal of the photodetector to perform calibration of the sensor assembly and coupled to the means for analyzing the amount of reflected light to detect the subject's blood oxygen concentration.

According to a further embodiment of the present disclosure, the light-emission means includes a plurality of banks of light emitting diodes.

According to a further embodiment of the present disclosure, the plurality of banks of light emitting diodes includes two banks emitting light respectively at approximately 850 nm, preferably 850 nm, and approximately 950 nm, preferably 950 nm.

According to a further embodiment of the present disclosure, the plurality of banks of light emitting diodes includes two banks emitting light respectively at approximately 600 nm, preferably 600 nm, and approximately 1100 nm, preferably 1100 nm.

According to a further embodiment of the present disclosure, the means for calibrating the light-emission means includes cycling through multiple wavelengths of light emitted by the light emission means to enable a spectral analysis of materials and oxy/deoxy-hemoglobin absorption to ascertain optimal wavelengths for material penetration and determination of oxygen saturation curves while maximally identifying movement and other artifacts.

According to a further embodiment of the present disclosure, the sensor assembly further comprises an input/output and processing means coupled to the light-receiver means to analyze the amount of reflected light to determine the blood oxygen concentration of the subject and/or to calibrate the light-emission means to take into consideration the presence of the at least one layer of clothing.

According to a further embodiment of the present disclosure, the input/output and processing means performs analysis of an output signal from the light-receiver means to perform calibration of the sensor assembly and detection and monitoring of the subject's blood oxygen concentration.

According to a further embodiment of the present disclosure, the input/output and processing means includes a communication bus that couples the light-receiver means and the light-emission means to the input/output and processing means.

According to a further embodiment of the present disclosure, the communication bus enables bidirectional communication to control emission of light by the light-emission means and receive reflected signals from the light-receiver means to perform processing for calibration, detection, and monitoring of the subject's blood oxygen content.

According to a further embodiment of the present disclosure, the input/output and processing means includes a processor.

According to a further embodiment of the present disclosure, a method for calibration and subsequent monitoring of a subject's blood oxygen concentration using the sensor assembly comprises the steps of detecting that a subject has come into contact with a sensor, emitting which light signals from a plurality of light emitting diode banks at at least one and potentially a plurality of particular wavelengths, detecting an amount of light that is reflected from the subject's blood, iteratively changing the emitted light emitting diode output light level up or down by smaller and smaller increments and analyzing, using a processor, a level of reflected light until an optimal emitted light emitting diode output light level is determined that produces an optimal reflected light level read by the photodiode, and monitoring the subject's blood oxygen concentration using the optimal emitted light emitting diode output light level.

According to a further embodiment of the present disclosure, while monitoring the subject's blood oxygen concentration, processing a signal indicating the detected amount of light reflected from the subject's blood through an operation amplifier and filter configuration to isolate a reflected light signal.

According to a further embodiment of the present disclosure, the filter configuration low-pass filters the signal at approximately 3 kHz, preferable 3 kHz.

According to a further embodiment of the present disclosure, the emitting of the light signals from the plurality of light emitting diode banks utilizes a set of light emitting diodes transmitting light at approximately 800 nm, preferably 800 nm, with another set of light emitting diodes transmitting light at a frequency selected to determine absolute blood volume, thereby creating a filter.

According to a further embodiment of the present disclosure, the processor analyzes the monitored blood oxygen concentration data to predict Fat Embolism Syndrome (FES), Hypoxemia, or a fracture complication.

According to a further embodiment of the present disclosure, calibration of the light-emission stage includes cycling through multiple wavelengths of light emitted by the light-emission means to enable a spectral analysis of materials and oxy/deoxy-hemoglobin absorption to ascertain optimal wavelengths for material penetration and determination of oxygen saturation curves while maximally identifying movement and other artifacts.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 provides a graph representing a relationship between a wavelength (nm) of light emitted from the sensor assembly and the specific absorption (1/cm·M), wherein the differences between the absorption spectra of deoxy- and oxy-hemoglobin is illustrated;

FIG. 2 is a top plan view of a sensor assembly in accordance with the present disclosure showing the sensor assembly next to a reference object to illustrate a size of the sensor assembly and that the sensor assembly includes various electronic components;

FIG. 3 is a bottom plan view of the sensor assembly of FIG. 2 showing the sensor assembly next to the reference object;

FIG. 4 is a schematic diagram showing electronic components included in a sensor assembly provided in accordance with the present disclosure;

FIG. 5 provides a table that includes additional information regarding the electronic components shown in FIG. 4; and

FIG. 6 is a diagrammatic view showing a flow chart for a methodology for calibrating and using the sensor assembly to monitor a subject's blood oxygen concentration.

FIG. 7 provides a schematic illustration of an oximetry signal generated using the disclosed embodiments.

FIG. 8 illustrates an example of extraction data for a pulse and breathing from an oximetry signal.

### DETAILED DESCRIPTION

Blood oxygen refers to the oxygen saturation of the blood and it is commonly referred to as SpO2 when it is measured by pulse oximetry, typically 94-98% in healthy adults. Oxygen saturation refers to oxygenation, or when oxygen molecules (O₂) enter the tissues of the human body. In the human body, blood is oxygenated in the lungs, where oxygen molecules travel from the air and into the blood. Oxygen saturation, or O₂ sats, is a measure of the percentage of hemoglobin binding sites in the bloodstream occupied by oxygen.

Measurement of a subject's oxygen saturation provides one indication of the subject's overall health and, more particularly, the subject's pulmonary and cardio-vascular health as both the pulmonary and cardio-vascular systems cooperate with each other and other systems of the human body to perform oxygenation.

Pulse oximetry, technically termed photo-plethysmography, is a conventional technique used in health care applications to monitor the heart beat, blood oxygenation as well as respiration rate and depth. The basic principal of pulse oximetry is the use of a sensor that operates by shining a light into a subject's tissue and measurement of how much light is absorbed versus transmitted. Because oxygenated blood and deoxygenated blood exhibit different absorption spectra, it is possible to determine the proportion of oxygenated blood, which increases each time a subject breaths and each time your heart pumps fresh blood through their arteries. Most common conventional pulse oximetry sensors monitor and detect the transmission of light through tissue (finger or ear lobe), while some look at light reflectance.

Arterial oxygenation is measured typically using pulse oximetry, which is a non-invasive technology for monitoring the saturation of a subject's hemoglobin. In transmissive pulse oximetry techniques, a sensor is placed on a thin part of a subject's body, for example, a fingertip or earlobe, or in the case of an infant, across a foot. Light of two different wavelengths is passed through the subject's tissue to a photodetector. The changing absorbance at each of the wavelengths is measured, allowing determination of the absorbances due to the pulsing arterial blood alone, excluding venous blood, skin, bone, muscle, and fat.

Another type of pulse oximetry is reflectance pulse oximetry. Reflectance pulse oximetry may be used as an alternative to transmissive pulse oximetry described above. Reflectance pulse oximetry does not require a thin section of a subject's body. Therefore, reflectance pulse oximetry is better suited to more universal application such as measurement of blood oxygen concentration in the feet, forehead, and chest. However, reflectance pulse oximetry also has some limitations.

For example, to date, academic literature has only recognized that reflectance pulse oximetry is possible. The literature has also recognized that there are issues with reliability and effect application of this technology. See, for example, “Photoplethysmography and its application in clinical physiological measurement,” John Allen (2007); “An Overview of Non-contact Photoplethysmography,” Peck Y S Cheang and Peter R Smith, 2012; “Contactless and continuous monitoring of heart rate based on photoplethysmography on a mattress,” M. Y. M. Wong, E. Pickwell-MacPherson and Y. T. Zhang, 2003; “Reflectance Pulse Oximetry Sensor for the Electronic Patch,” Rasmus G. Haahr, November 2006; “A Novel Method for the Contactless and Continuous Measurement of Arterial Blood Pressure on a Sleeping Bed,” W. B. Gu, C. C. Y. Poon, H. K. Leung, M. Y. Sy, M. Y. M. Wong and Y. T. Zhang, 2009; and “Wavelength Selection Method with Standard Deviation: Application to Pulse Oximetry,” Vaquez-Jaccaud_et_al, 2011. Each of these references is incorporated herein by reference in its entirety.

Thus, embodiments disclosed herein have been developed to identify and address issues of calibration that have, until now, been obstacles to development of an operational and reliable, reflectance pulse oximetry sensor that self calibrates to the subject being monitored and, in particular, the numbers and types of layer(s) of clothing being worn by the subject. In doing so, disclosed embodiments of the sensor assembly identify optimum level(s) and type(s) of emitted light to provide meaningful and reliable pulse oximetry data for a subject. Because each subject may have a different amount and type of clothing through which the emitted light must travel and/or reflect, proper functioning of the sensor assembly uses automated calibration of the sensor assembly on a per session/subject basis. That is, every time that a subject comes in contact with the sensor, recalibration is performed for the sensor assembly(ies) to account for the possibility that a new subject may be monitored and/or that the subject's clothing layers may have been changed in type or quantity. This innovation in accordance with the present disclosure has significant utility in a wide variety of applications as explained herein.

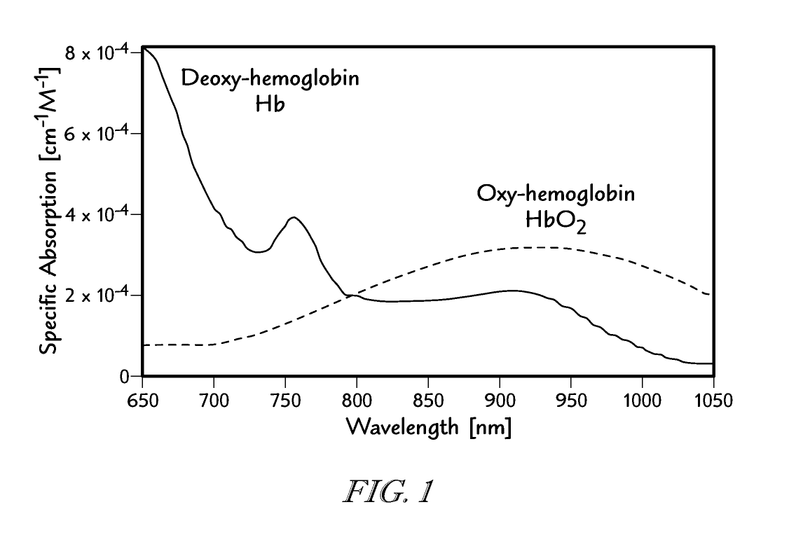

By way on introduction of the disclosed embodiments, pulse oximetry is based on the principal that oxy- and deoxy-hemoglobin have different light absorption spectra as shown in FIG. 1. Reflective pulse oximetry measuring the light absorption of light of two different wavelengths via reflectivity; that is, by knowing the amount of light transmitted and detecting the amount of light reflected using a photodector or similar sensor, one is able to determine the amount of light absorbed by the subject's body, i.e., the light absorption.

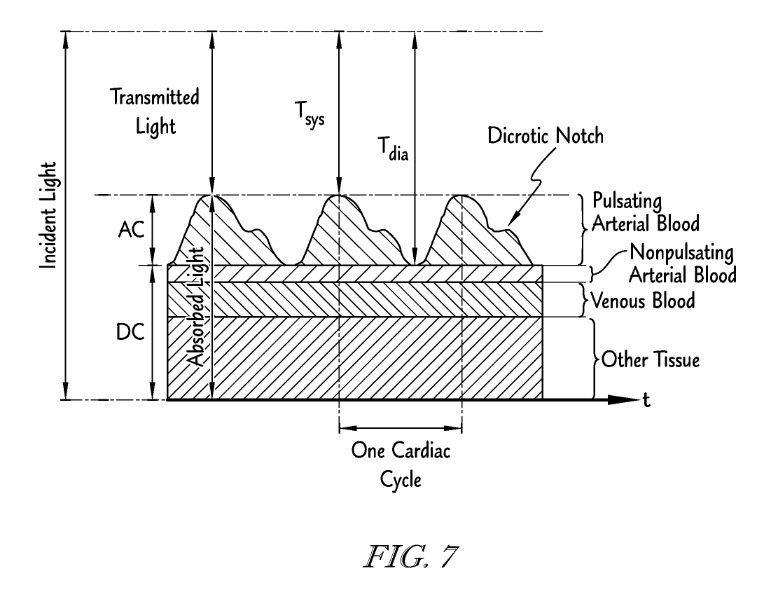

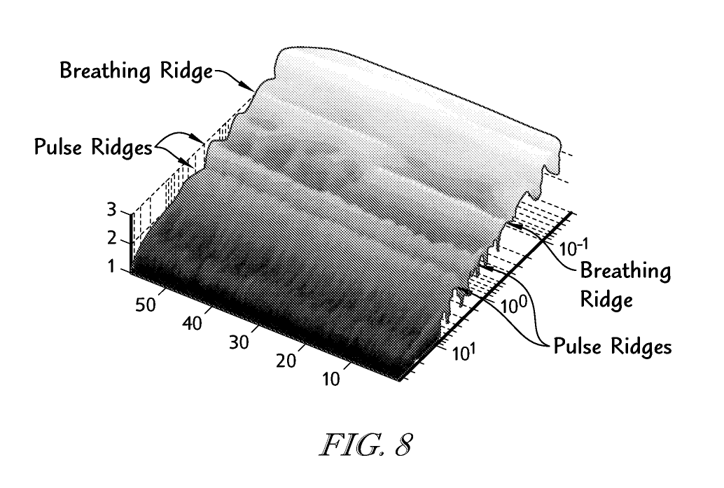

FIG. 7 provides a schematic illustration of a pulse oximetry signal. FIG. 8 illustrates an example showing extraction of pulse and breathing information from such a pulse oximetry signal.

However, the efficacy of non-contact pulse oximetry through intervening materials is subject to the absorption spectra of those materials. To create an oximetry (e.g., PulseOx) sensor that is able to determine blood oxygenation through a variable makeup of intervening materials, the disclosed embodiments provide the ability to switch between or select from multiple wavelengths of light to be transmitted at the subject's body. Based on the reflected amount of light resulting from the various wavelengths, the sensor assembly is able to select one or more optimum wavelengths of light to be transmitted at the subject's body to determine the oxygen saturation for the subject via reflective pulse oximetry.

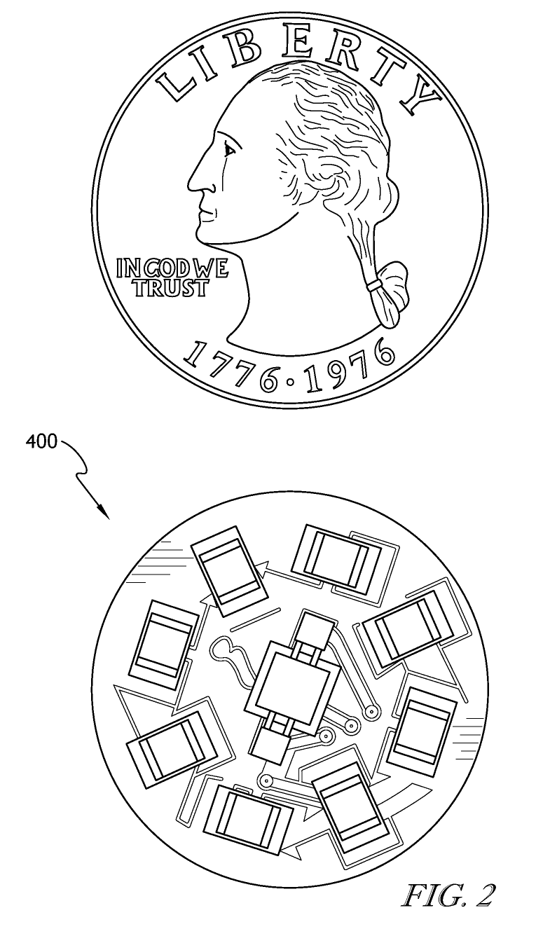

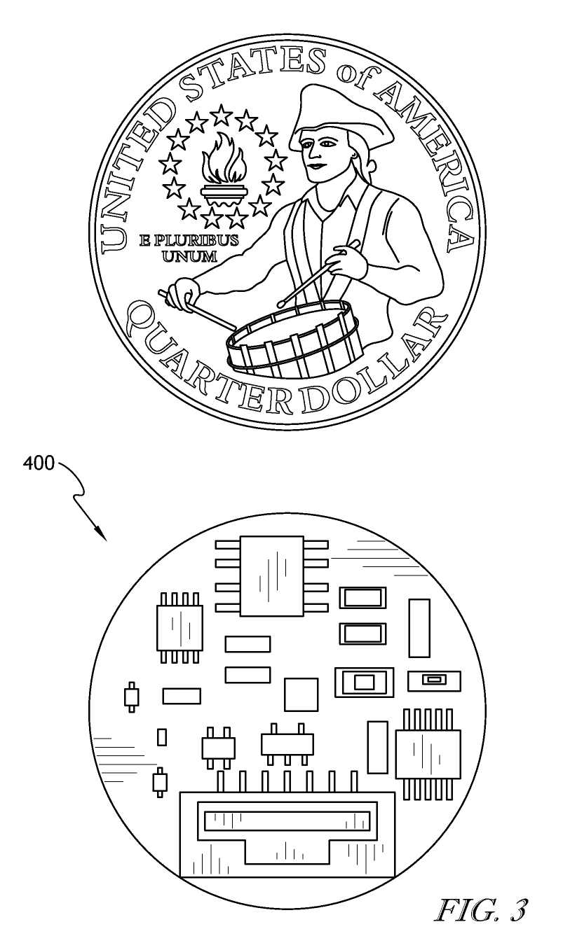

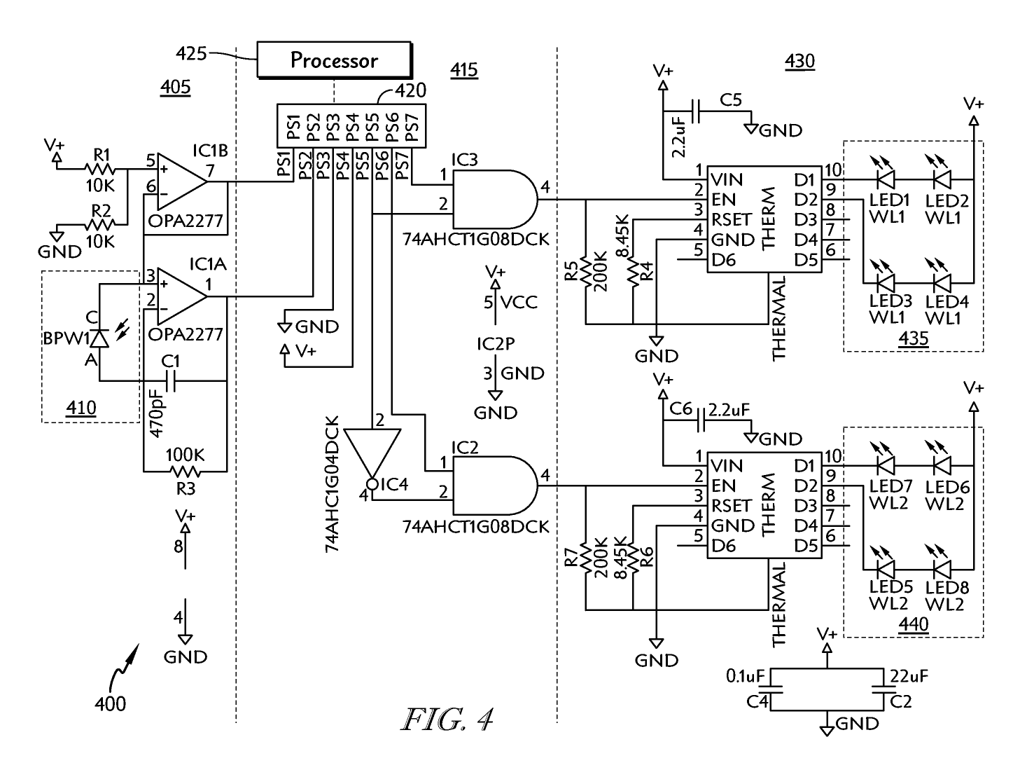

FIGS. 2 and 3 provide a top plan view and bottom plan view respectively of electronic components included in a sensor assembly 400 (illustrated in schematic in FIG. 4) provided in accordance with disclosed embodiments. Both views are shown in relation to a reference object, here a U.S. quarter, to illustrate a reference size.

FIG. 4 is a schematic diagram illustrating electronic components of a sensor assembly provided in accordance with disclosed embodiments. As shown in FIG. 4, at least one disclosed embodiment of the sensor assembly 400 includes three stages: a photodetector stage 405, an input/output and processing stage 415 and a light emission stage 430.

The photodetector stage 405 includes the photodetector or photodiode 410 that is used to detect reflected amounts of light from the subject's body. The photodetector stage 405 also includes various circuitry elements that enable buffering and filtering of the detected signal including operational amplifiers for establishing a virtual ground and buffering and filtering of the signal output from the photodetector 410.

The teachings of U.S. Pat. No. 5,348,004, entitled “Electronic Processor for Pulse Oximeter” and U.S. Pat. No. 6,839,580, entitled “Adaptive Calibration for Pulse Oximetry” are both hereby incorporated by reference herein in their entirety. Each of those patents disclose various equipment, components, and methodology that may be used to implement the disclosed embodiments for sensing and monitoring blood oxygen in a seating environment.

The output of the photodetector stage 405 is coupled to the input/output and processing stage 415 so as to enable analysis of the signal detected by the photodetector to perform calibration of the sensor assembly and detection and monitoring of the subject's blood oxygen content. The input/output and processing stage 415 includes a communication bus 420 that couples the sensor assembly components of stages 405 and 430 with the processor 425. This coupling and associated bidirectional communication enables the processor 425 to control emission of light via the light emission stage 430 and receive reflected signals from the photodetector stage 405 to perform processing for calibration, detection, and monitoring of the subject's blood oxygen content.

The light emission stage 430 includes two banks of LEDs, 435, 440. The LED banks may be optimized to use off-the-shelf LEDs at, for example, 850 nm and 950 nm light that penetrate a wide range of materials well. The light emission stage 430 may use additional or alternative banks of LEDs, for example, at additional wavelengths between 600 nm and 1100 nm for greater robustness of signal to noise determination.

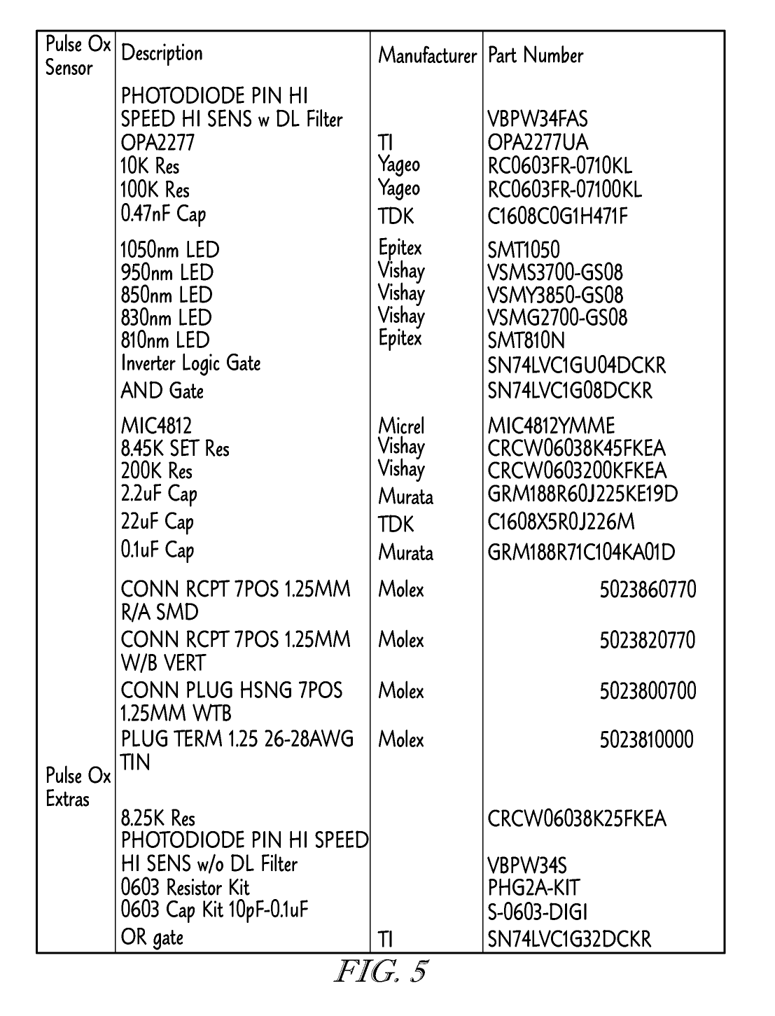

In implementation, the stages illustrated in FIG. 4 and the incorporated components are selected from commercially available electronics components listed in the table of FIG. 5. Further, it should be noted that the photodiode 410, i.e. the receptor, and the LEDs of the LED banks 435, 440, i.e., the emitter, may be approximately 7.5 mm to avoid spill over from the LEDs to the photodiode

Embodiments disclosed herein provide the ability to perform non-invasive, non-distracting monitoring of blood oxygen contact through multiple layers of material. A calibration sub-routine for sensor and sensor assembly learns the best light components for a particular subject being monitored. This is because the light components used for reflective monitoring change depending on the amount, type, and number of clothing layers for a particular subject.

Thus, disclosed embodiments may use custom designed circuitry developed to read PulseOx (also known as photoplesythmography, or PPG) signals through variable layers of intervening clothing worn by a subject. Thus, disclosed embodiments enable sensor assembly calibration cycling through multiple wavelengths of light to enable a spectral analysis of materials and oxy/deoxy-hemoglobin absorption to ascertain optimal wavelengths for material penetration and determination of oxygen saturation curves while maximally identifying movement and other artifacts.

Disclosed embodiments of the sensor assembly perform auto-calibration, which enables the ability to penetrate an unknown makeup of intervening material to read changes in reflected light that accompany fluctuations in oxy- and deoxy-hemoglobin accompanying each heartbeat. Because some of the relevant aspects of PulseOx signals change at very slow time-scales (e.g., respiration changes 10+ seconds), simply using high-pass filtering of the signal merely creates substantial distortions and delays. To avoid the problems of high-pass filters, custom circuitry and algorithms were developed.

Disclosed embodiments include circuitry that may use high-frequency pulse-width modulation to adjust the output light level of the LEDs. However, several other methods are possible, including a digitally controlled potentiometer.

Thus, disclosed embodiments integrate prior art technology and improve upon it to provide a PulseOx sensor that can operate effectively through clothing and/or seat trim and provide methodology to effectively calibrate sensor and integrate with custom hardware/software for analysis of collected data.

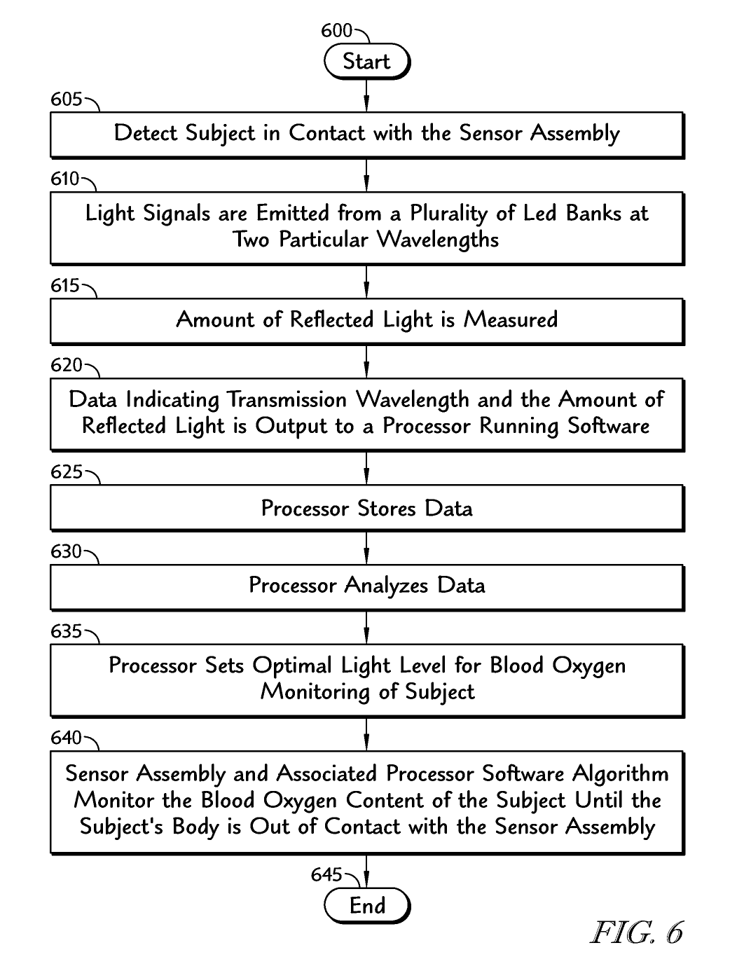

As illustrated in FIG. 6, a methodology for performing calibration and subsequent monitoring of a subject's blood oxygen concentration is performed using the above-described sensor assembly. The methodology begins at 600 and control proceeds to 605 at which it is detected that a subject has come into contact with the sensor assembly. Control then proceeds to 610 at which light signals are emitted from a plurality of LED banks at at least one and potentionally a plurality of particular wavelengths. Certain clothing material may have unique absorption spectra that make transmission of different wavelengths an effective option. Also, one potential option may be two utilize a set of LEDs at approximately 800 nm (crossover point for oxy and deoxy) with another set of LEDs for use in determining absolute blood volume so as to enable creation of a highly accurate filter.

Subsequent to transmission, the amount of light that is reflected from the subject's blood is measured by the photodetector/photodiode included in the sensor assembly and the signal is then fed through an operation amplifier and filter configuration to isolate the reflected light signal at 615. For example, the signal may be low-pass filtered at approximately 3 kHz to improve the ability to isolate the signal.

Data in the form of an analog or digital signal indicating the transmission wavelength and the amount of reflected light is then output at 620 to a processor running software that stores the data at 625 and analyzes the data 630. The processor's software analyzes the data at 630 by analyzing the reflected light resulting from iteratively changing the emitted LED output light level up or down by smaller and smaller increments until an optimal reflected light level is read by the photodiode. One example of software instructions provided to implement this calibration is as follows (written in the language C for ease of understanding; however, the software instructions may be written in any programming language that has some degree of utility):

```
for (1=1; i<nIterations; i++)
  photoLevel = readFrom(photodiode);
  if (photoLevel < minLevel) {
      pwmDuty = pwmDuty + (0.65 / i);
  } else if (photoLevel > maxLevel) {
      pwmDuty = pwmDuty − (0.65 / i);
  }
    wait(iterationDelay);
}
```

Subsequently, the optimal light levels to be emitted by the LED bank(s) is set by operation 635. Control then proceeds to operation 640, at which the sensor assembly and associated processor software algorithm monitor the blood oxygen content of the subject until the subject's body is out of contact with the sensor assembly and operations end at 645.

In order to improve robustness of data detected by the oximetry sensor, more than one sensor may be used to monitor a subject's blood oxygen content. Thus, two sensor assemblies of the type illustrated in FIG. 4 would be used to monitor a particular subject. A critical assumption about biosensor data gathered from non-clinical settings is that the data from any (or all) sensors may be unreliable at any given point in time. To address this reality, each signal is given a reliability score. This reliability score may be used both to assess the trustworthiness of the particular signal as well as way to combine multiple estimates of, e.g. heart rate, into a single heart rate metric that has greater reliability than any of the heart rates calculated from individual sensors. This approach leads to substantial gains in robustness of the system through redundancy.

Any type of biosensor data gathered from non-clinical settings may be subject to error as a result that data from any (or all) sensors may be unreliable at any given point in time. To address this reality, providing multiple sensors to check, confirm, and compare data increases the robustness of a system for monitoring subject well-being as a result of redundancy.

In such an implementation, each of the sensor assemblies may be included in a seat upon which a subject is sitting, for example, a vehicle seat within an automobile. In such an application, the processor 425 used in the input/output and processing stage 415 could be specific to each sensor assembly or shared by a plurality of sensor assemblies. Moreover, the processor 425 may be implemented within the automotive vehicle's computing system.

Moreover, at least some components of the sensor assembly 400, for example, processor 425, may also be used to control other types of sensors provided in a subject's seat. Such sensors may permit gathering and analysis of complimentary biometric data to be analyzed regarding the subject's well being, for example, Electro Cardio Gram (ECG) signal monitoring. In fact, both ECG and PulseOx sensors both offer redundant heart rate information, but also offer complimentary biometric information including Heart Rate Variability (HRV) from the ECG signal and respiration information from the PulseOx signal. Thus, the presently disclosed embodiments may be used with other types of sensors to provide complimentary information that can be used via various specialized algorithms running on the processor 425 to gather a more full understanding of a subject's health and wellness state.

Accordingly, looking at the combined data from multiple sensors including the presently disclosed PulseOx sensor assembly provides not only the sum of the individual metrics, but emergent interaction effects in a “whole is greater than the sum” manner. Thus, for example, using the PulseOx sensor assembly in combination with ECG monitoring enables the ability to calculate peak times of an ECG signal and the PulseOx signal separately; by looking at the time difference between the two signals, the processor algorithm can ascertain a Pulse Transit Time (PTT, possibly more accurately Pulse Arrival Time, PAT). Research has shown that this PTT is highly correlated with blood pressure. See C. Douniama, C. U. Sauter, R. Couronne, “Blood Pressure Tracking Capabilities of Pulse Transit Times in Different Arterial Segments: A Clinical Evaluation,” Computers in Cardiology 2009; 36: 201-204.

Whereas a subject's blood pressure may increase, the time it takes for blood to get from a subject's heart to the PulseOx sensor through the subject's arteries decreases. Thus, information provided by and ECG sensor (which monitors the changes in electrical potential of the body as a result of heart activity) and the presently disclosed PulseOx sensor (which measures changes of the level of oxygen in the blood as a result of heart activity and respiration) provides new opportunities to investigate and analyze the health and wellness of a subject through simultaneous gathering of complimentary but related data.

The data generated by the presently disclosed sensor assembly may be written into a text file that may include a plurality of previous sessions of monitoring. The sensor assembly may be used to acquire live signals and the processor (and/or other processors) may be used to perform trend analysis if the sensors are used in an implementation where the same subject is monitored repeatedly.

The output of the sensor assembly may be coupled to an alarm that indicates whether a subject's blood oxygen content has fallen below an acceptable level. Such an implementation may have significant utility for monitoring subject's with pulmonary issues such Chronic Obstructed Pulmonary Disease, lung cancer, asthma, etc. By implementing the sensor in a pad that may be placed on a wheel chair or within the wheel chair itself, a subject sitting in the chair may be monitored without cumbersome wires attached to the subject's body, e.g., those associated with transmissive PulseOx sensors described above.

While the presently disclosed innovation has been described in conjunction with the specific embodiments outlined above, it is evident that many alternatives, modifications, and variations will be apparent to those skilled in the art. Accordingly, the various disclosed embodiments, as set forth above, are intended to be illustrative, not limiting. Various changes may be made without departing from the spirit and scope of the invention.

For example, although the presently disclosed embodiments have been disclosed in the context of monitoring blood oxygen content in an automotive environment, disclosed embodiments may be used in any number of different applications and environments that would benefit from the utility afforded by persistent, effortless contact sensing via the disclosed sensor assembly. For example, the disclosed embodiments may be used in seating for office, home and specialized seating, e.g., video game, theater, amusement ride seating, etc. For such implementations, utility may be provided by using the sensor alone or in combination with other sensors to monitor various physiological parameters of a subject to better understand status, emotion, response to stimulus, etc.

By detecting the emotional responses of a seat occupant, the oximetry sensor seating can provide feedback that is sensitive to the emotional context. Accordingly, using oximetry sensors, alone or in combination with other types of sensors in this way enables the development of safety features beyond traditional reactive safety systems to predict unsafe states to avert hazards before they present a dangerous scenario. Features like drowsiness warning or a medical attack alert could present substantial value to improve safety.

Prototype design and analysis utilized LabView as the single point of control, but commercial implementation may utilize a higher level of autonomy. For example, the pulse oximetry sensor and associated software may be configured to auto-calibrate to a seat occupant's clothing; thus, in one implementation, the pulse oximetry sensor may receive and response to a signal indicating when a new person has sat down and thereby perform an auto-calibration routine without the need to communicate with a centralized CPU that communicates with other sensors in a seat and coordinates the sensor operation and data generation and analysis.

Thus, in accordance with a disclosed embodiment, the pulse oximetry sensor may include the ability to penetrate an unknown makeup of intervening clothing material to read the changes in reflected light that accompany the fluctuations in oxy- and deoxy-hemoglobin accompanying each heartbeat. Because some of the relevant aspects of oximetry signals change at very slow time-scales (10+ seconds, e.g. respiration), simply high-pass filtering the signal may create substantial distortions and delays. To avoid the problems of high-pass filters custom circuitry and algorithms may be developed using high-frequency pulse-width modulation to adjust the output light level of the LEDs; however, several other methods are possible, including use of a digitally controlled potentiometer, e.g., picking up the signal reflected by the blood is a photodiode and operational amplifier, low-pass filtered at ˜3 kHz. Thus, an associated calibration control algorithm may iteratively change the LED output light level up or down by smaller and smaller jumps until an optimal reflected light level is read by the photodiode, as explained above with regard to software code.

Alternatively, a pulse oximetry sensor module may include a built-in, microcontroller that handles both LED bank switching logic as well as the auto-calibration logic.

The sensor assembly may have particular use in hospital beds, wheel chairs, and other devices in contact with amputees for predicting Fat Embolism Syndrome (FES), Hypoxemia, and other fracture complications (see Dr. Deepanjali Pant, Dr. Narani K. K., Dr. Vijay Vohra, Dr. Jayashree Sood, “EARLY DIAGNOSIS OF EVOLVING FAT EMBOLISM SYNDROME BY SIMPLE PULSE OXIMETRY IN HIGH-RISK PATIENTS,” Indian J. Anastaesth. 2006; 50(6):463-465.

Based on the research of Pant et al. relating to monitoring of amputees using pulse ox sensors to predict those at high risk of FES and other related amputation complications), disclosed embodiments of the sensor assembly in accordance with the present disclosure could be incorporated into equipment incorporated into any piece of equipment that comes into contact with an amputee to provide data that may be used to better monitor the health and well-being of these subjects and provide early detection of amputation complications.

In accordance with the embodiments disclosed herein, the sensor and/or sensor assembly can be re-calibrated for each new monitoring (e.g., sitting, laying or other positioned setting the results in persistent, effortless contact) session for the sensor(s).

Thus, the sensor assembly has utility in a host of potential applications including hospital beds, wheel chairs, infant bedding, and incubator equipment, just as a few examples. Thus, the ability to measure and monitor blood oxygen contact in a non-invasive, effortless way in accordance with the present disclosure has many more applications than the implementation in an automotive vehicle environment. Accordingly, the embodiments disclosed herein have potential applications including ergonomics, rehabilitation, and the detection of illnesses which affect the blood circulation (e.g., peripheral vascular disease). The disclosed sensor assembly has potential application including the monitoring of blood oxygen concentration in subjects who are being monitored for various conditions that do not enable accurate monitoring of blood oxygen concentration with conventional pulse oximetry techniques. For example, subjects suffering from various heart conditions, for example, congenital cyanotic heart disease patients, can suffer a combination of arterial and venous pulsations in the forehead region that lead to spurious SpO₂ (Saturation of peripheral oxygen) results.

Likewise, position of a subject's body in the Trendelenburg position, wherein the subject's body is laid flat on the back (supine position) with the feet higher than the head by 15-30 degrees have been known to result in inaccurate pulse oxygen monitoring using convention oximetry technology. Nevertheless, the Trendelenburg position is a standard position used in abdominal and gynecological surgery because it allows better access to the pelvic organs as gravity pulls the intestines away from the pelvis. Thus, the presently disclosed sensor assemblies in accordance with the present disclosure may be implemented in a surgical table or pad placed upon a surgical table upon which such patients are placed during such surgeries.

Further, the sensors and/or sensor assemblies may be implemented in back boards, gurneys and/or stretchers for use in transporting injured or ill subjects. By implementing the sensor/sensor assembly in the horizontal plane upon which the subject is resting, the sensor/sensor assembly is able to monitor the blood oxygen content of the subject without requiring external wires and a cumbersome set up. This may have particular utility in rescue and trauma equipment such as back boards, where medical personnel are attempting to tend to a subject's immediate needs and must delay setting up extensive monitoring of the patients vital signs. Because the disclosed sensor assembly is able to provide persistent, effortless and self calibrating detection and monitoring of a subject, the quality of care resulting from use of the equipment including such an assembly is significantly increased.

The sensor/sensor assembly may communicate with diagnostic equipment that is located in close proximity and/or remote proximity to the horizontal surface to provide data regarding blood oxygen concentration to those medical personnel that need the information to provide proper care and treatment. To this end, the sensor assembly may incorporate, communicate with and/or use fiber optic technology that enables remote sensing. Thus, in at least some disclosed applications, the photodetector sensor is itself an optical fiber. In other potential implements, fiber may be used to connect a non-fiber optic sensor to a measurement system. Depending on the application, fiber may be used because of its small size, or the fact that no electrical power is required at the remote location, or because many sensors can be multiplexed along the length of a fiber by using different wavelengths of light for each sensor. Thus, the sensor assembly could be implemented in whole or in part using fiber optic technology. A particularly useful feature of such fiber optic sensors is that they can, if required, provide distributed sensing over distances of up to one meter.

Various connections are set forth between elements in the following description; however, these connections in general, and, unless otherwise specified, may be either direct or indirect, either permanent or transitory, and either dedicated or shared, and that this specification is not intended to be limiting in this respect.

The functionality described in connection with various described components of various embodiments in accordance with the present disclosure may be combined or separated from one another in such a way that the architecture of the present disclosure is somewhat different than what is expressly disclosed herein. Unless otherwise specified, there is no essential requirement that methodology operations be performed in the illustrated order; therefore, one of ordinary skill in the art would recognize that some operations may be performed in one or more alternative order and/or simultaneously.

Various components of the present disclosure may be provided in alternative combinations operated by, under the control of or on the behalf of various different entities or individuals.

Further, it should be understood that, in accordance with at least one embodiment of the present disclosure, system components may be implemented together or separately and there may be one or more of any or all of the disclosed system components. Further, system components may be either dedicated systems or such functionality may be implemented as virtual systems implemented on general purpose equipment via software implementations.

Unless otherwise expressly stated, it is in no way intended that any operations set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following inventive concepts.

As a result, it will be apparent for those skilled in the art that the illustrative embodiments described are only examples and that various modifications can be made within the scope of the present disclosure

1. A sensor assembly for measuring a subject's blood oxygen concentration, the sensor assembly comprising
light-emission means for emitting light at least one wavelength, the light reflecting from the subject's body through at least one layer of clothing,
light-receiver means for receiving the reflected light from the subject's body,
means for analyzing the amount of reflected light to determine the blood oxygen concentration of the subject, and
means for calibrating the light-emission means to take into consideration the presence of the at least one layer of clothing.

2. The sensor assembly of claim 1, wherein the means for analyzing the amount of reflected light determines an amount of light absorbed by the subject's body to determine the blood oxygen concentration of the subject.

3. The sensor assembly of claim 2, wherein the means for analysing the amount of reflected light detects the blood oxygen concentration to predict Fat Embolism Syndrome (FES), Hypoxemia, or a fracture complication.

4. The sensor assembly of claim 3, wherein the means for analyzing the amount of reflected light from the subject's body does so using reflectance pulse oximetry.

5. The sensor assembly of claim 4, wherein the means for calibrating the light-emission means identifies at least one optimum level and/or at least one type of emitted light for emission by the light-emission means.

6. The sensor assembly of claim 1, wherein the means for calibrating the light-emission means performs calibration every time that a subject comes in contact with a sensor located in the seating environment in the motor vehicle.

7. The sensor assembly of claim 1, wherein the light-receiver means is a photodetector that measures an amount of light reflected from the subject's body.

8. The sensor assembly of claim 7, further comprising circuitry coupled to the photodetector for buffering and filtering of a photodetector output signal and at least one operational amplifier that establishes a virtual ground and buffering and filtering of the photodetector output signal.

9. (canceled)

10. The sensor assembly of claim 8, wherein the output signal of the photodetector is coupled to the means for calibrating the light-emission means and performs analysis of the output signal of the photodetector to perform calibration of the sensor assembly and coupled to the means for analyzing the amount of reflected light to detect the subject's blood oxygen concentration.

11. The sensor assembly of claim 1, wherein the light-emission means includes a plurality of banks of light emitting diodes.

12. The sensor assembly of claim 11, wherein the plurality of banks of light emitting diodes includes two banks emitting light respectively at approximately 850 nm, preferably 850 nm, and approximately 950 nm, preferably 950 nm.

13. The sensor assembly of claim 12, wherein the plurality of banks of light emitting diodes includes two banks emitting light respectively at approximately 600 nm, preferably 600 nm, and approximately 1100 nm, preferably 1100 nm.

14. The sensor assembly of claim 13, wherein the means for calibrating the light-emission means includes cycling through multiple wavelengths of light emitted by the light emission means to enable a spectral analysis of materials and oxy/deoxy-hemoglobin absorption to ascertain optimal wavelengths for material penetration and determination of oxygen saturation curves while maximally identifying movement and other artifacts.

15. The sensor assembly of claim 1, further comprising an input/output and processing means coupled to the light-receiver means to analyze the amount of reflected light to determine the blood oxygen concentration of the subject and/or to calibrate the light-emission means to take into consideration the presence of the at least one layer of clothing.

16. (canceled)

17. The sensor assembly of claim 15, wherein the input/output and processing means includes a communication bus that couples the light-receiver means and the light-emission means to the input/output and processing means.

18. The sensor assembly of claim 17, wherein the communication bus enables bidirectional communication to control emission of light by the light-emission means and receive reflected signals from the light-receiver means to perform processing for calibration, detection, and monitoring of the subject's blood oxygen content.

19. The sensor assembly of claim 19, wherein the input/output and processing means includes a processor.

20. A method for calibration and subsequent monitoring of a subject's blood oxygen concentration using a sensor assembly of any preceding claim, the method comprising the steps of
detecting that a subject has come into contact with a sensor,
emitting which light signals from a plurality of light emitting diode banks at at least one and potentially a plurality of particular wavelengths,
detecting an amount of light that is reflected from the subject's blood,
iteratively changing the emitted light emitting diode output light level up or down by smaller and smaller increments and analyzing, using a processor, a level of reflected light until an optimal emitted light emitting diode output light level is determined that produces an optimal reflected light level read by the photodiode, and
monitoring the subject's blood oxygen concentration using the optimal emitted light emitting diode output light level.

21. The method of claim 20, while monitoring the subject's blood oxygen concentration, processing a signal indicating the detected amount of light reflected from the subject's blood through an operation amplifier and filter configuration to isolate a reflected light signal.

22. The method of claim 21, wherein the filter configuration low-pass filters the signal at approximately 3 kHz, preferable 3 kHz.

23. The method of claim 22, wherein the emitting of the light signals from the plurality of light emitting diode banks utilizes a set of light emitting diodes transmitting light at approximately 800 nm, preferably 800 nm, with another set of light emitting diodes transmitting light at a frequency selected to determine absolute blood volume, thereby creating a filter.

24. The method of claim 23, wherein the processor analyzes the monitored blood oxygen concentration data to predict Fat Embolism Syndrome (FES), Hypoxemia, or a fracture complication.

25. (canceled)
